(19) United States
(12) Patent Application Publication
(10) Pub. No.: US 2022/0025464 A1
(43) Pub. Date: Jan. 27, 2022

(54) METHODS AND MATERIALS FOR DETECTING GENE COPY NUMBER VARIANTS

(71) Applicant: GOPATH LABORATORIES LLC, Buffalo Grove, IL (US)

(72) Inventors: Jim LU, Buffalo Grove, IL (US); Zhongmin GUO, Buffalo Grove, IL (US)

(21) Appl. No.: 17/281,716

(22) PCT Filed: Sep. 27, 2019
(86) PCT No.: PCT/US19/53523

§ 371 Date: Mar. 31, 2021

Related U.S. Application Data
(60) Provisional application No. 62/739,573, filed on Oct. 1, 2018.

(51) Int. Cl. C12Q 1/6886

(52) U.S. Cl. CPC C12Q 1/6886

(57) ABSTRACT

The disclosure provides next generation sequencing-based methods and materials for detecting a gene copy number variant in a biological sample having one or more genes. The disclosure also provides an electronic computer system for detecting a gene copy number variant. Detection of gene copy number variants may be used to enable patients with increased risks associated with certain diseases to take preventative measures to reduce their risk or receive targeted treatment to improve their chances of survival.

### CROSS REFERENCE TO RELATED APPLICATIONS

The present application claim priority to and the benefit of U.S. Provisional Patent Application Ser. No. 62/739,573, filed Oct. 1, 2018, which is incorporated herein by reference in its entirety.

### FIELD

The present disclosure generally provides methods and materials for detecting a gene copy number variant in a biological sample having one or more genes. The present disclosure also provides an electronic computer system for detecting a gene copy number variant.

### BACKGROUND

Hereditary cancers are a major concern for patients with a family history of cancer. Clinical genetic testing to detect genetic variants and, in particular, gene copy number variants (CNVs), associated with a risk for cancer can be a powerful tool by informing patients whether they have an increased risk of cancer. Patients with an increased cancer risk can take preventative measures to lower their cancer risk and can also undergo routine screening and detection procedures. By doing so, these patients can reduce their risk of cancer and can improve their survival chances by early detection and targeted treatment. Unfortunately, current methods for screening gene CNVs are time consuming and labor intensive, particularly when next generation sequencing (NGS) is incorporated. Additionally, conventional methods are able to detect base substitutions, small insertions and deletions, but have had difficulty detecting duplications and deletions of CNVs due to the short-read sequencing data available on most NGS platforms.

For example, hereditary breast and ovarian cancer syndrome (HBOCS) represents up to 10% of all breast cancers diagnosed annually. A number of genes are associated with hereditary breast and ovarian cancer susceptibility, including BRCA½, TP53, PTEN, and CDH1. Foulkes, W. D., Inherited susceptibility to common cancers, 359(20) N. Engl. J. Med. 2143, 2143-53 (2008). Of those genes, BRCA½ confer a risk of breast cancer that is 10 to 30 times higher than the general population. Antoniou, A., et al., Average risks of breast and ovarian cancer associated with BRCA1 or BRCA2 mutations detected in case series unselected for family history: a combined analysis of 22 studies, 72(5) Am. J. Hum. Genet. 1117, 1117-30 (2003). Patients with CNVs in any gene associated with hereditary cancer and, in particular, patients with CNVs in BRCA1/2 would benefit greatly from accurate clinical genetic testing.

In fact, clinical genetic testing for hereditary cancers, such as HBOCS, is recommended. Such testing can identify germline gene mutations, such as CNVs, in patients with a family history of cancer, providing those patients with the option to take preventative measures, as discussed above. For patients identified as having germline gene mutations related to HBOCS, they can opt to have risk-reducing procedures, such as salpiingo-oophorectomy, and receive targeted therapy, such as the use of PARP inhibitors. Daly, M. B., et al., NCCN Guidelines Insights: Genetic/Familial High-Risk Assessment: Breast and Ovarian, Version 2.2017, 15(1) Natl. Compr. Canc. Netw. 9, 9-20 (2017). Similar risk-reducing procedures and targeted therapies are also available for patients that have germline gene mutations associated with other hereditary cancers.

Several methods are currently available to detect CNVs, including multiplex ligation-dependent amplification (MLPA), multiplex amplifiable probe hybridization (MAPH), and array-based comparative genome hybridization (aCGH). See, Kousoulidou, L., et al., Multiple Amplifiable Probe Hybridization (MAPH) methodology as an alternative to comparative genomic hybridization (CGH), 653 Methods Mol. Biol. 47, 47-71 (2010); Ceulemans, S., et al., Targeted screening and validation of copy number variations, 838 Methods Mol. Biol. 311, 311-28 (2012); Eijk-Van Os, P.G., et al., Multiplex Ligation-dependent Probe Amplification (MLPA(R)) for the detection of copy number variation in genomic sequences, 688 Methods Mol. Biol. 97, 97-126 (2011). However, incorporating these methods into an NGS workflow has been found to be both time consuming and labor intensive. Because of the high-throughput capability and affordable cost of NGS multigene panels, there is a strong desire to incorporate them into methods for CNV detection. Thus, there is a need for methods that can accurately detect CNVs using NGS more quickly and more cost effectively than traditional methods. Further, there is a need for such methods that can accurately and reliably detect CNVs as small as a single exon variation.

### SUMMARY

The present disclosure relates to methods and materials for detecting gene CNVs. The present disclosure also relates to an electronic computer system for detecting gene CNVs.

The present disclosure provides methods and materials for detecting a gene CNV in a biological sample having one or more genes, including next generation sequencing-based methods for detecting a gene CNV. The methods may comprise: obtaining a set of probes for NGS wherein each probe in the set hybridizes a different segment of the one or more genes; performing NGS with the set of probes on the biological sample comprising the one or more genes to obtain a sequence read for each probe; creating a normalization baseline for a probe; generating a coverage index for a probe in the set of probes; and determining a difference between the coverage index of the probe and a set confidence interval established from a normal range of coverage index, wherein a CNV is detected where a p-value for the difference is equal to or less than a set threshold. In this manner, the methods disclosed herein may be used to detect a cancer risk for a patient, including an increased cancer risk.

In some embodiments of each or any of the above or below mentioned embodiments, the methods further comprise obtaining the biological sample from a patient. In some embodiments, the biological sample is a liquid biopsy. In some embodiments, the liquid biopsy is an aspirate, blood, plasma, serum, sputum, urine, or saliva. In a preferred embodiment, the liquid biopsy is a blood sample. In some embodiments, the biological sample is a solid tumor. In some embodiments, the biological sample is a fresh tissue sample.

In some embodiments of each or any of the above or below mentioned embodiments, the set of probes obtained for NGS comprise probes that each hybridize a different segment of the one or more genes. The probes may each hybridize different segments of 1, 2, 3, 4, 5, 6, 7, 8, 9, 10, 15, 20, 25, 30, 35, 40, 45, 50, or more genes. In some embodiments, the probes may hybridize different segments of genes associated with cancers. In some embodiments, the probes may hybridize different segments of genes associated with diseases or disorders that are linked to germline or somatic genetic CNV. In a preferred embodiment, the probes hybridize different segments of genes associated with breast and ovarian cancer. In a particularly preferred embodiment, the probes hybridize different segments of at least 15 genes associated with hereditary breast and ovarian cancer syndrome. In some embodiments, the probes hybridize different segments of overlapping regions of exons, or exon-intron boundaries. In a preferred embodiment, the probes hybridize overlapping regions of exons and exon-intron boundaries of at least 15 genes, including at least 15 genes associated with hereditary breast and ovarian cancer syndrome. In a particularly preferred embodiment, the probes hybridize overlapping regions of exons and exon-intron boundaries of at least 15 genes, including BRCA1, BRCA2, APC, MLH1, MSH2, MSH6, PMS2, EPCAM, TP53, CDH1, STK11, SMAD4, VHL, NF2, PTEN, ATM, AXIN2, BARD1, BLM, BMPR1A, BRIP1, BUB1B, CDK4, CDKN2A, CHEK2, EXO1, FLCN, GREM1, MLH3, MRE11A, MUTYH, NBN, NF1, PALB2, PMS1, POLD1, POLE, RAD50, RAD51C, RAD51D, and TGFBR2.

In some embodiments of each or any of the above or below mentioned embodiments, the normalization baseline for each probe is based on the total number of sequence reads for the set of probes. In some embodiments, the normalization baseline is calculated by adding the sequence reads from each probe to obtain the total number of sequence reads for the set of probes for the biological sample and dividing the total number of sequence reads for the biological sample by the number of probes in the set of probes.

In some embodiments of each or any of the above or below mentioned embodiments, the coverage index of a probe is determined by dividing the number of sequence reads obtained for the probe by the normalization baseline.

In some embodiments of each or any of the above or below mentioned embodiments, the normal range of coverage index is determined by obtaining one or more biological samples having a normal copy number for each of the one or more genes; performing NGS on the one or more biological samples with the set of probes; adding the sequence reads from each probe to obtain a total number of sequence reads for the set of probes for each of the biological samples; dividing the total number of sequence reads for each of the biological samples by the number of probes in the set of probes to generate a normalization baseline for a probe; calculating a coverage index for each probe in the set of probes for the biological sample by dividing the number of sequence reads obtained for the probe by the normalization baseline; calculating an established mean and an established standard deviation for each probe using the coverage indices for the probes; and establishing a confidence interval for each probe using the established mean and the established standard deviation. In some embodiments, the confidence level is 55%, 60%, 65%, 70%, 75%, 80%, 85%, 90%, 95%, or 99%. In a preferred embodiment, the confidence level is 99%.

In some embodiments, if the probability value is equal to or less than 10⁻², 10 −3, or 10⁻⁴, the biological sample has a CNV for the gene covered by the probe. In other embodiments, the set threshold is 10⁻⁴.

In some embodiments of each or any of the above or below mentioned embodiments, the CNV may be an exon, intron, duplication (amplification), or deletion. In some embodiments, the deletion may be heterozygous or homozygous.

The present disclosure also provides methods and materials for detecting a gene CNV in a biological sample having one or more genes. The methods may comprise: obtaining a set of probes for next generation sequencing wherein each probe in the set hybridizes a different segment of the one or more genes in the biological sample; performing next generation sequencing with the set of probes on the biological sample to obtain a sequence read for each probe; adding the sequence reads from each probe to obtain a total number of sequence reads for the set of probes; dividing the total number of sequence reads by the number of probes in the set of probes to generate a normalization baseline for a probe; determining a coverage index for the probe in the set of probes by diving a number of sequence reads obtained for the probe by the normalization baseline; and generating a p-value for a difference between the coverage index of the probe and a set confidence interval established from a normal range of coverage index, wherein a CNV is detected where the p-value is equal to or less than 10⁻², 10⁻³, or 10⁻⁴.

The present disclosure further provides an electronic computer system that may comprise one or more processors; and a memory storing one or more programs for execution by the one or more processors, the one or more programs comprising instructions for: analyzing data obtained from next generation sequencing of a biological sample having one or more genes using a set of probes, wherein the data comprises sequence reads for each probe; creating a normalization baseline for a probe; generating a coverage index for a probe in the set of probes; determining a difference between the coverage index of the probe and a set confidence interval established from a normal range of coverage index, wherein a copy number variant is detected where a p-value for the difference is equal to or less than a set threshold.

### BRIEF DESCRIPTION OF THE FIGURES

FIGS. 1A-1B are normalized graphs of the results from the detection of a CNV in the MSH6 gene in a biological sample. FIG. 1A shows the results where duplications in the copy number of the MSH6 gene were detected. FIG. 1B shows the results where a normal range of copy numbers of the MSH6 gene were detected.

FIGS. 2A-2B are normalized graphs of the results from the detection of a CNV in the MSH2 gene in a biological sample. FIG. 2A shows the results where deletions in the copy number of the MSH2 gene were detected. FIG. 2B shows the results where a normal range of copy numbers of the MSH2 gene were detected.

FIG. 3 is a table of data from probes used to detect CNVs in the MSH6 gene. The table shows data from a sample containing a normal copy number of the MSH6 gene and from an abnormal sample containing duplications of the copy number of the MSH6 gene.

FIG. 4 is a table of data from probes used to detect CNVs in the MSH2 gene. The table shows data from a sample containing a normal copy number of the MSH2 gene and from an abnormal sample containing deletions of the copy number of the MSH2 gene.

### DETAILED DESCRIPTION

Methods are provided herein for detecting a gene copy number variant in a biological sample having one or more genes. Such methods may detect gene copy number variants associated with an increased risk of cancer using next generation sequencing. Consequently, the methods may overcome the challenges associated with incorporating next generation sequencing into conventional methods for detecting CNVs. Additionally, the CNVs detected with the methods provided herein may be used as a basis to take preventative measures to reduce a patient's cancer risk and to perform early, routine cancer screening.

The present disclosure provides methods and materials for detecting a gene CNV in a biological sample having one or more genes. The methods may comprise: obtaining a set of probes for NGS wherein each probe in the set hybridizes a different segment of the one or more genes (e.g., genes associated with HBOCS); performing NGS with the set of probes on the biological sample (e.g., a blood sample) comprising the one or more genes to obtain a sequence read for each probe; creating a normalization baseline for a probe; generating a coverage index for a probe in the set of probes (e.g., by dividing the number of sequence reads obtained for the probe by the normalization baseline); and determining a difference between the coverage index of the probe and a set confidence interval established from a normal range of coverage index, wherein a CNV is detected where a p-value for the difference is equal to or less than a set threshold. In an embodiment, the normal range of coverage index is determined by obtaining one or more biological samples having a normal copy number for each of the one or more genes; performing NGS on the one or more biological samples with the set of probes; adding the sequence reads from each probe to obtain a total number of sequence reads for the set of probes for each of the biological samples; dividing the total number of sequence reads for each of the biological samples by the number of probes in the set of probes to generate a normalization baseline for a probe; calculating a coverage index for each probe in the set of probes for the biological sample by dividing the number of sequence reads obtained for the probe by the normalization baseline; calculating an established mean and an established standard deviation for each probe using the coverage indices for the probes; and establishing a confidence interval for each probe using the established mean and the established standard deviation.

The methods and materials for detecting a gene CNV in a biological sample having one or more genes may also comprise: obtaining a set of probes for next generation sequencing wherein each probe in the set hybridizes a different segment of the one or more genes in the biological sample (e.g., genes associated with HBOCS); performing NGS with the set of probes on the biological sample to obtain a sequence read for each probe; adding the sequence reads from each probe to obtain a total number of sequence reads for the set of probes; dividing the total number of sequence reads by the number of probes in the set of probes to generate a normalization baseline for a probe; determining a coverage index for the probe in the set of probes by diving a number of sequence reads obtained for the probe by the normalization baseline; and generating a p-value for a difference between the coverage index of the probe and a set confidence interval established from a normal range of coverage index, wherein a CNV is detected where the p-value is equal to or less than 10⁻⁴.

The methods provided herein may additionally comprise administering a targeted therapy to a patient if a CNV is detected in his or her biological sample.

The present disclosure further provides an electronic computer system that comprises one or more processors; and a memory storing one or more programs for execution by the one or more processors, the one or more programs comprising instructions for: analyzing data obtained from next generation sequencing of a biological sample having one or more genes using a set of probes, wherein the data comprises sequence reads for each probe; creating a normalization baseline for a probe; generating a coverage index for a probe in the set of probes; determining a difference between the coverage index of the probe and a set confidence interval established from a normal range of coverage index, wherein a copy number variant is detected where a p-value for the difference is equal to or less than a set threshold.

### Definitions

As used herein, “normal range of coverage index” refers to a database or collection of data comprising information reflecting normal copy numbers for one or more genes. The index is generated based on information obtained from next generation sequencing (NGS) of biological samples using a set of probes wherein each probe in the set hybridizes a different segment of one or more genes. The segments can cover specific regions of a gene, or exons and exon-intron boundaries of one or more genes. The biological samples are obtained from patients known to have normal copy numbers for the one or more genes. That is, the patients have no deletions or duplications in the copy number of the one or more genes. The index can be generated from 1, 5, 10, 15, 20, 25, 50, 75, 100, or more biological samples. In a preferred embodiment, the database is generated from at least 100 biological samples. NGS is performed on the biological samples using the set of probes to obtain a sequence read for each probe. A normalization baseline is calculated from this information by adding the sequence reads from each probe in the set to obtain a total number of sequence reads for the set of probes and dividing the total number of sequence reads by the number of probes in the set of probes. The normalization baseline is used to calculate the coverage index for each probe, which is calculated by dividing the total number of sequence reads obtained for the probe by the normalization baseline.

As used herein, “established mean” refers to a mean calculated by adding the coverage indices for each probe and dividing by the total number of probes in the set of probes.

As used herein, “established standard deviation” refers to a value calculated using the established mean.

As used herein, “confidence interval” refers to a range of values defined so that there is a specified probability, also referred to as the confidence level, that the value of a parameter lies within it. Here, the confidence interval is calculated for each probe using the established mean and established standard deviation calculated from the normal range of coverage index using the following formula: Confidence interval=(established mean−2.57*established standard deviation, established mean+2.57*established standard deviation). The confidence interval can be based on a confidence level of 55%, 60%, 65%, 70%, 75%, 80%, 85%, 90%, 95%, or 99%. In a preferred embodiment, the confidence level is 99%.

As used herein, “normalization baseline” refers to a baseline value that is calculated by adding the sequence reads from each probe to obtain a total number of sequence reads for the set of probes and dividing the total number of sequence reads by the number of probes in the set of probes.

As used herein, “coverage index” refers to a value for a probe that is calculated by dividing a number of sequence reads obtained for the probe by the normalization baseline.

As used herein, the “probability value” or “p-value” is a value that is calculated for each probe based on the coverage index and the established mean and established standard deviation from the normal range of coverage index using the following equation: p value=2*(1-NORMSDIST(ABS(coverage index—established mean)/established standard deviation)). If the probability value is equal to or less than 10⁻⁴, the biological sample has a CNV for the gene covered by the probe.

### Detection of a Gene CNV in a Biological Sample

Provided herein are methods of detecting gene CNVs in biological samples having one or more genes that can be used to assess a patient's risk of developing cancer. Surprisingly, the inventors found that NGS could be used with a set of probes covering various regions of genes known to be associated with an inherited susceptibility to cancer (e.g., overlapping regions of exons, exon-intron boundaries, and the like) to generate a coverage index for each probe containing information from which a CNV in a particular gene can be accurately and reliably detected. The detection of a CNV can be used to inform a patient of their cancer risk and provide them with the opportunity to take steps to mitigate their risk and/or to have themselves closely monitored for the occurrence of cancer. In this manner, the methods disclosed herein provide a personalized approach to detecting whether a patient has a hereditary risk of cancer.

The present disclosure also provides methods and materials for detecting a gene CNV in a biological sample having one or more genes. The methods may comprise: obtaining a set of probes for NGS wherein each probe in the set hybridizes a different segment of the one or more genes (e.g., genes associated with HBOCS); performing NGS with the set of probes on the biological sample (e.g., a blood sample) comprising the one or more genes to obtain a sequence read for each probe; creating a normalization baseline for a probe; generating a coverage index for a probe in the set of probes (e.g., by dividing the number of sequence reads obtained for the probe by the normalization baseline); and determining a difference between the coverage index of the probe and a set confidence interval established from a normal range of coverage index, wherein a CNV is detected where a p-value for the difference is equal to or less than a set threshold.

The present disclosure further provides methods and materials for detecting a gene CNV in a biological sample having one or more genes may also comprise: obtaining a set of probes for next generation sequencing wherein each probe in the set hybridizes a different segment of the one or more genes in the biological sample (e.g., genes associated with HBOCS); performing NGS with the set of probes on the biological sample to obtain a sequence read for each probe; adding the sequence reads from each probe to obtain a total number of sequence reads for the set of probes; dividing the total number of sequence reads by the number of probes in the set of probes to generate a normalization baseline for a probe; determining a coverage index for the probe in the set of probes by diving a number of sequence reads obtained for the probe by the normalization baseline; and generating a p-value for a difference between the coverage index of the probe and a set confidence interval established from a normal range of coverage index, wherein a CNV is detected where the p-value is equal to or less than 10⁻⁴, 10⁻³, or 10⁻⁴.

A set of probes for next generation sequencing may be obtained based on the one or more genes for which a CNV is desired to be detected. The set of probes may comprise 10, 15, 20, 25, 30, 35, 40, 45, 50, 55, 60, 65, 70, 75, 80, 85, 90, 95, 100, 125, 150, 175, 200, 250, 300, 350, 400, 450, 500, 550, 600, 650, 700, 750, 800, 850, 900, 950, 1000, 1050, 1100, or more individual probes. In a preferred embodiment, the set of probes comprises over 1050 individual probes. The probes may be created to hybridize different segments of one or more genes associated with a known risk of cancer, such as genes associated with breast and ovarian cancer. The probes may hybridize different segments of those genes, such as overlapping regions of exons, or exon-intron boundaries. The different segments may be of 1, 2, 3, 4, 5, 6, 7, 8, 9, 10, 15, 20, 25, 30, 35, 40, 45, 50, or more genes. The set of probes may be created using known methodologies, such as IDT in-silico technology.

The biological sample may be blood or saliva. Additionally, the provided methods may further comprise obtaining the biological sample from a patient. The biological sample may be obtained from a liquid biopsy, which may be an aspirate, blood, plasma, serum, sputum, urine, or saliva. Preferably, the liquid biopsy is a blood sample or saliva sample. The biological sample may also be a fresh tissue sample. The biological sample may also be a solid tumor.

Genomic DNA may be extracted from the biological sample using well-known conventional methods. A threshold amount of genomic DNA may be required for the disclosed methods, such as NGS. The threshold amount of genomic DNA can be 1 ng, 5 ng, 10 ng, 15 ng, 20 ng, 25 ng, 30 ng, 35 ng, 40 ng, 45 ng, 50 ng, 55 ng, 60 ng, 65 ng, 70 ng, 75 ng, 80 ng, 85 ng, 90 ng, 95 ng, 100 ng, 1 μg, 2 μg, 3 μg, 4 μg, 5 μg, 6 μg, 7 μg, 8 μg, 9 μg, 10 μg, 15 μg, 20 μg, 25 μg, or 30 μg.

Next generation sequencing is performed on the biological sample comprising one or more genes using the set of probes using known methodologies. From the next generation sequencing, a sequence read is obtained for each probe. Thus, the next generation sequencing provides the number of sequence reads for each probe as well as the aggregate number of sequence reads for the set of probes.

A normalization baseline is created for a probe. Such a normalization baseline may be created for each probe. The normalization baseline may be calculated by adding the number of sequence reads from each probe to obtain the total number of sequence reads for the set of probes and dividing that total by the number of probes in the set of probes. The normalization baseline may be used to generate a coverage index for a probe in the set of probes. A coverage index may be created for each probe. The normalization baseline and coverage index may be used to normalize the sequence read data obtained from next generation sequencing.

The normal range of coverage index may be determined by obtaining one or more biological samples having a normal copy number for each of the one or more genes; performing NGS on the one or more biological samples with the set of probes; adding the sequence reads from each probe to obtain a total number of sequence reads for the set of probes for each of the biological samples; dividing the total number of sequence reads for each of the biological samples by the number of probes in the set of probes to generate a normalization baseline for a probe; calculating a coverage index for each probe in the set of probes for the biological sample by dividing the number of sequence reads obtained for the probe by the normalization baseline; calculating an established mean and an established standard deviation for each probe using the coverage indices for the probes; and establishing a confidence interval for each probe using the established mean and the established standard deviation.

The normal range of coverage index may be used to establish a set confidence interval. The normal range of coverage index may comprise information reflecting normal copy numbers for one or more genes (e.g., normal copy numbers for genes associated with HBOCS). The normal range of coverage index may be generated based on information obtained from NGS of biological samples known to have normal copy numbers of the one or more genes. Normal copy numbers of the one or more genes are copy numbers where there are no deletions are duplications. A set of probes wherein each probe in the set hybridizes a different segment of the one or more genes may be used to perform NGS. The segments may cover specific regions of a gene, or exons and exon-intron boundaries of one or more genes. The normal range of coverage index may be generated from information obtained from 1, 5, 10, 15, 20, 25, 50, 75, 100, or more biological samples. In one embodiment, the database is generated from at least 100 biological samples. NGS is used to obtain the number of sequence reads for each probe. That information is used to calculate a normalization baseline, which is done by adding the sequence reads from each probe to obtain a total number of sequence reads for the set of probes and dividing the total number of sequence reads by the number of probes in the set of probes. The normalization baseline is used to calculate the coverage index for each probe by dividing the total number of sequence reads obtained for the probe by the normalization baseline. The coverage index is used to calculate an established mean by adding the coverage indices for each probe and dividing by the total number of probes in the set of probes. The established mean is used to calculate an established standard deviation. The established mean and established standard deviation are used to calculate a set confidence interval.

A difference between the coverage index of a probe and a set confidence interval may be determined by calculating a p-value for the difference. The p-value may be calculated based on the coverage index, the established mean, and the established standard deviation. A CNV is detected where the p-value is equal to or less than a set threshold. The set threshold may be 10⁻⁴.

A detected CNV may be an exon, intron, duplication (amplification), or deletion. The deletion may be heterozygous or homozygous.

Method of Treating a Patient with a Targeted Therapy

The present disclosure also provides methods and materials for treating a patient with a targeted therapy. The method may comprise determining if a gene copy number variant (CNV) is present in a biological sample obtained from the patient, which comprises the steps of: obtaining a set of probes for next generation sequencing (NGS) wherein each probe in the set hybridizes a different segment of the one or more genes, performing next generation sequencing with the set of probes on the biological sample comprising the one or more genes to obtain a sequence read for each probe, creating a normalization baseline for a probe, generating a coverage index for a probe in the set of probes, and determining a difference between the coverage index of the probe and a set confidence interval established from a normal range of coverage index, wherein a CNV is detected where a p-value for the difference between the coverage index of the probe and the set confidence interval is equal to or less than a set threshold; and administering the targeted therapy to the patient where a CNV is detected in the biological sample.

### Electronic Computer System for Detecting a CNV

The present disclosure provides an electronic computer system that may comprise one or more processors; and a memory storing one or more programs for execution by the one or more processors, the one or more programs comprising instructions for: analyzing data obtained from next generation sequencing of a biological sample having one or more genes using a set of probes, wherein the data comprises sequence reads for each probe; creating a normalization baseline for a probe; generating a coverage index for a probe in the set of probes; determining a difference between the coverage index of the probe and a set confidence interval established from a normal range of coverage index, wherein a copy number variant is detected where a p-value for the difference is equal to or less than a set threshold.

The coverage index of the probe may be determined by dividing the number of sequence reads obtained for the probe by the normalization baseline.

The normal range of coverage index may be determined by obtaining one or more biological samples having a normal copy number for each of the one or more genes; performing NGS on the one or more biological samples with the set of probes; adding the sequence reads from each probe to obtain a total number of sequence reads for the set of probes for each of the biological samples; dividing the total number of sequence reads for each of the biological samples by the number of probes in the set of probes to generate a normalization baseline for a probe; calculating a coverage index for each probe in the set of probes for the biological sample by dividing the number of sequence reads obtained for the probe by the normalization baseline; calculating an established mean and an established standard deviation for each probe using the coverage indices for the probes; and establishing a set confidence interval for each probe using the established mean and the established standard deviation. The set confidence interval may be based on a 99% confidence level. The set threshold may be 10⁻², 10⁻³, or 10⁻⁴.

### EXAMPLES

### Example 1

### Detection of CNVs Associated with Hereditary Breast and Ovarian Cancer Syndrome

Biological samples were obtained from patients, some of whom were known to lack CNVs associated with certain breast and/or ovarian cancer related genes. A total of 121 samples were obtained. The biological samples were either peripheral blood or saliva samples. DNA was extracted from the samples using known methodologies. Approximately 100 ng of genomic DNA was obtained from each sample. DNA libraries for NGS were created from the samples using a KAPA HyperPlus Library Prep Kit from Kapa Biosystems and following the manufacturer's protocol. KAPA Library Quantification Kits were used in accordance with the manufacturer's protocol for quality control.

A set of probes for NGS was designed and synthesized using Integrated DNA Technologies' in-silico technology. The set of probes was designed to hybridize 1,070 overlapping segments of exons and exon-intron boundaries of 15 genes, including BRCA1, BRCA2, APC, MLH1, MSH₂, MSH6, PMS2, EPCAM, TP53, CDH1, STK11, SMAD4, VHL, NF2, and PTEN. The set of probes contained 554 probes.

The DNA libraries from each sample were pooled and loaded onto an Illumina®MiSeq system at a molarity of 12 pM. A 151 paired-end dual index was run using an Illumina® MiSeq Reagent Kit v2. MiSeq Reporter software was used to generate a FASTQ file containing the sequence read for each probe. NextGENe® software by Softgenetics® was used to perform secondary and tertiary analyses of the initial NGS results obtained from the MiSeq system. The secondary and tertiary analysis included the generation of a mutation report that provided coverage data for each probe.

The sequence read data was used to create a normalization baseline for a probe. The normalized baseline was calculated using software that added the number of sequence reads from each probe to obtain the total number of sequence reads for the set of probes that was used. This value was divided by 554, the total number of probes in the set of probes, to obtain the normalization baseline. The normalization baseline was used to create a coverage index for each of the 554 probes. The coverage index was created for each probe by dividing the number of sequence reads obtained for the probe by the normalization baseline. From this data, a p-value was generated for a difference between the coverage index of the probe and a set confidence interval established from a normal range of coverage index.

A normal range of coverage index was determined from 79 of the 121 samples, which were negative for the BRCA1 and BRCA2 exon CNVs. These samples underwent multiplex ligation-dependent amplification to analyze the BRCA1 and BRCA2 exons using kits and protocols from MRC Holland. All 79 samples were confirmed to be negative for BRCA1 and BRCA2 CNVs. NGS was performed on these 79 samples in accordance with the methodologies described above to generate a normalization baseline and a coverage index for each probe. An established mean and an established standard deviation were calculated for each probe using the coverage indices. The established mean was calculated by adding the coverage indices for each probe and dividing by the total number of probes in the set of probes, here 554. The established mean was then used to calculate an established standard deviation. The established mean and established standard deviation were used to calculate a set confidence interval. The confidence interval was calculated based on a 99% confidence level.

The difference between the coverage index of the probe, discussed above, and the set confidence interval was determined and a p-value was calculated. Here, a CNV was detected where the p value was less than 0.01.

Of the 42 samples that were not used to determine a normal range of coverage index, 14 samples were positive for exon CNVs in MLH1, MSH2, MSH6, PMS2, BRCA2, CHEK2, and BARD1. The 28 other samples were negative for CNVs and were previously tested as negative for BRCA1 and BRCA2 CNVs by multiplex ligation-dependent probe amplification.

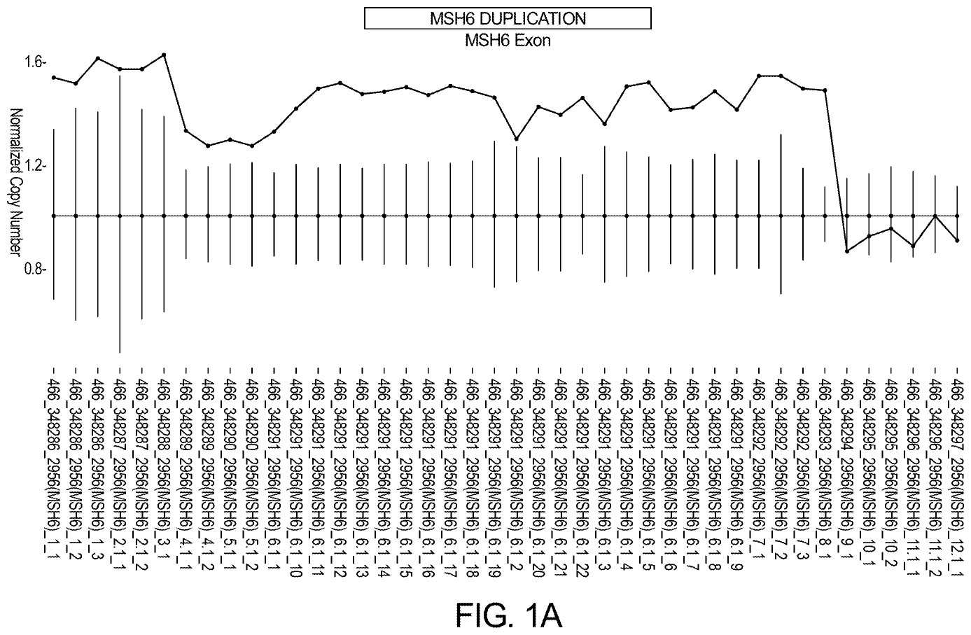

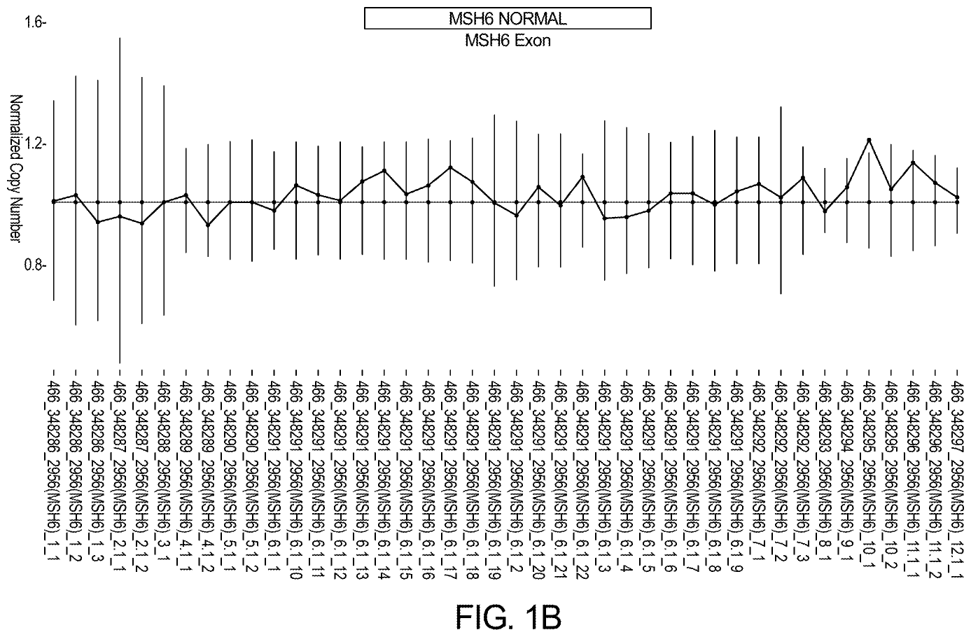

FIGS. 1A-1B are normalized graphs showing the results from the detection of a CNV in the MSH6 gene in a biological sample using the disclosed methods. The x-axis contains information regarding the probes used to cover the MSH6 exon and thereby indicates the position on the exon. The y-axis corresponds to the normalized copy number of the MSH6 gene contained in the biological sample. The bars on the graph indicate the normal range of copy numbers from the MSH6 gene. Any point or line falling outside of that range indicates an abnormally high or low number of MSH6 gene copy numbers. FIG. 1A shows the results from a biological sample that contained duplications in the copy number of the MSH6 gene. These results could indicate that the patient has an increased risk for hereditary breast and ovarian cancer syndrome. FIG. 1B comparatively shows the results from a biological sample that contained a normal range of copy numbers of the MSH6 gene.

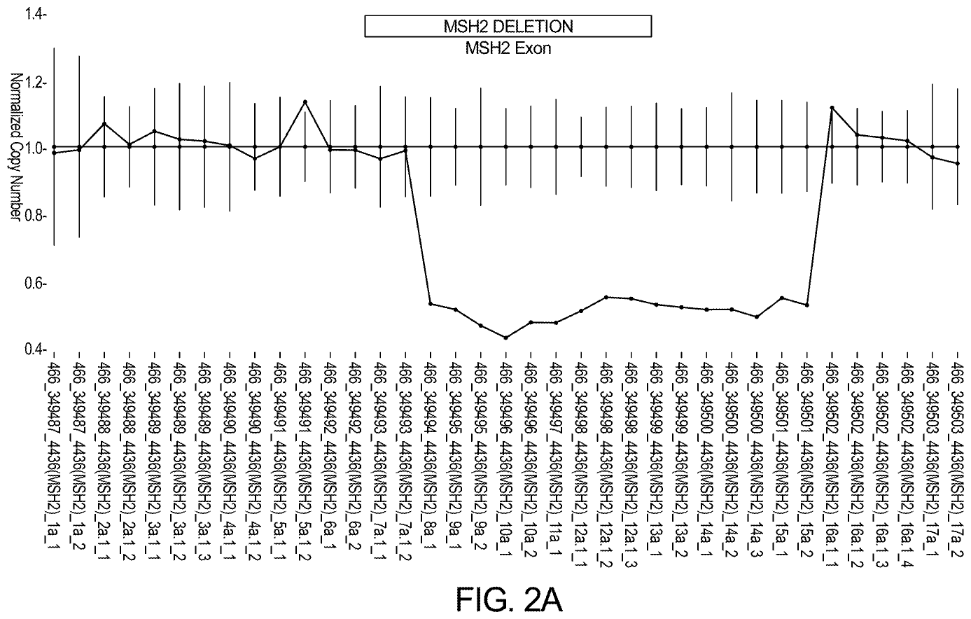

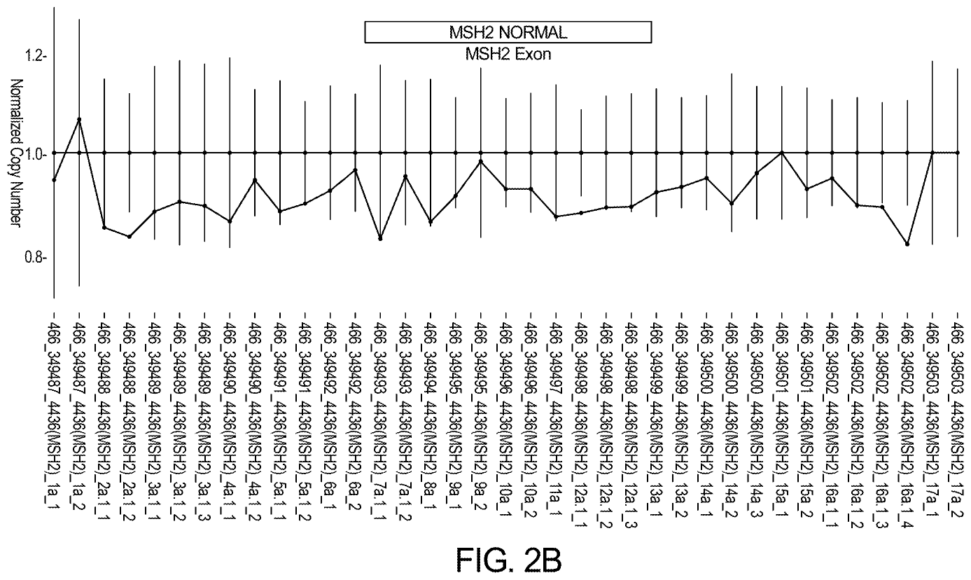

FIGS. 2A-2B are normalized graphs showing the results from the detection of a CNV in the MSH2 gene in a biological sample using the disclosed methods. The x-axis contains information regarding the probes used to cover the MSH2 exon and thereby indicates the position on the exon. The y-axis corresponds to the normalized copy number of the MSH2 gene contained in the biological sample. The bars on the graph indicate the normal range of copy numbers from the MSH2 gene. Any point or line falling outside of that range indicates an abnormally high or low number of MSH2 gene copy numbers. FIG. 2A shows the results from a biological sample that contained deletions in the copy number of the MSH2 gene. These results could indicate that the patient has an increased risk for hereditary breast and ovarian cancer syndrome. FIG. 2B comparatively shows the results from a biological sample that contained a normal range of copy numbers of the MSH2 gene.

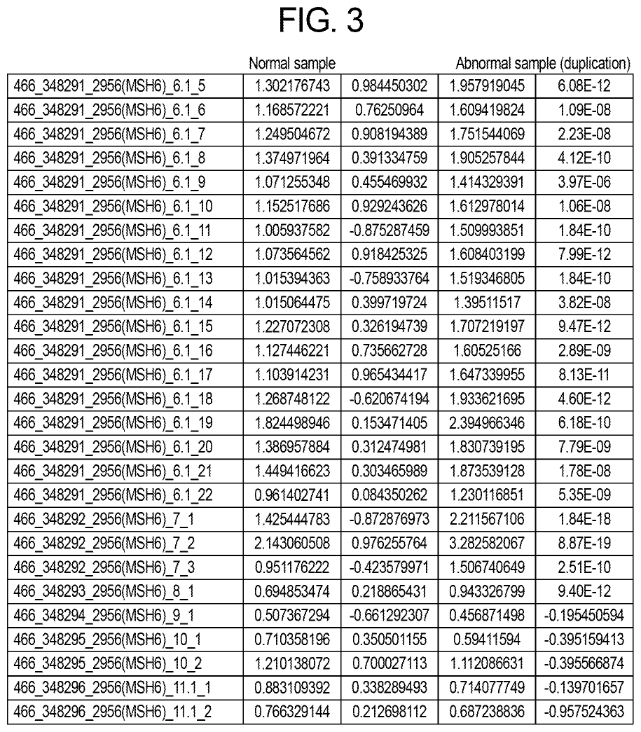

FIG. 3 is a table of data from the probes used to detect CNVs in the MSH6 gene. This data shows the results from the disclosed NGS and analysis of a biological sample containing a normal copy number of the MSH6 gene and from a biological sample containing duplications of the copy number of the MSH6 gene. The left hand column for each sample contains the normalized copy number of the MSH6 gene and the right hand column for each sample contains the normalized standard deviation. The standard deviation numbers that are shaded in the abnormal sample columns correspond to the detected CNV duplications.

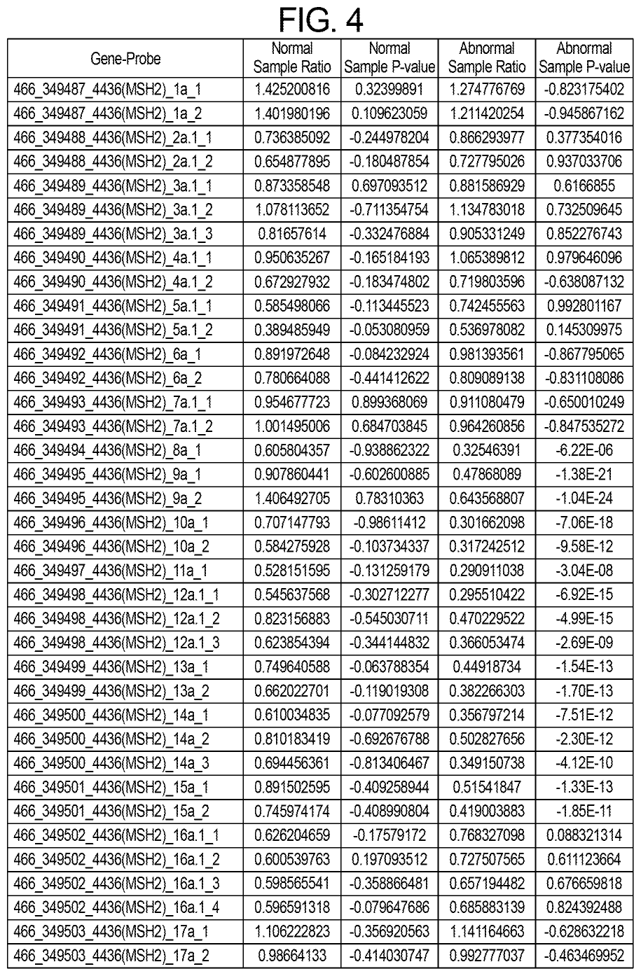

FIG. 4 is a table of data from the probes used to detect CNVs in the MSH2 gene. This data shows the results from the disclosed NGS and analysis of a biological sample containing a normal copy number of the MSH2 gene and from a biological sample containing deletions of the copy number of the MSH2 gene. The left hand column for each sample contains the normalized copy number of the MSH2 gene and the right hand column for each sample contains the normalized standard deviation. The standard deviation numbers that are shaded in the abnormal sample columns correspond to the detected CNV duplications.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements. The terms “a,” “an,” “the” and similar referents used in the context of describing the disclosure (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the disclosure and does not pose a limitation on the scope of the disclosure otherwise claimed. No language in the specification should be construed as indicating any non-claimed element essential to the practice of the disclosure.

Groupings of alternative elements or embodiments of the disclosure disclosed herein are not to be construed as limitations. Each group member can be referred to and claimed individually or in any combination with other members of the group or other elements found herein. It is anticipated that one or more members of a group can be included in, or deleted from, a group for reasons of convenience and/or patentability. When any such inclusion or deletion occurs, the specification is deemed to contain the group as modified, thus fulfilling the written description of all Markush groups used in the appended claims.

Certain embodiments of this disclosure are described herein, including the best mode known to the inventor for carrying out the disclosure. Of course, variations on these described embodiments will become apparent to those of ordinary skill in the art upon reading the foregoing description. The inventor expects skilled artisans to employ such variations as appropriate, and the inventor intends for the disclosure to be practiced otherwise than specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

Specific embodiments disclosed herein can be further limited in the claims using “consisting of” or “consisting essentially of” language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the disclosure so claimed are inherently or expressly described and enabled herein.

It is to be understood that the embodiments of the disclosure disclosed herein are illustrative of the principles of the present disclosure. Other modifications that can be employed are within the scope of the disclosure. Thus, by way of example, but not of limitation, alternative configurations of the present disclosure can be utilized in accordance with the teachings herein. Accordingly, the present disclosure is not limited to that precisely as shown and described.

While the present disclosure has been described and illustrated herein by references to various specific materials, procedures and examples, it is understood that the disclosure is not restricted to the particular combinations of materials and procedures selected for that purpose.

Numerous variations of such details can be implied as will be appreciated by those skilled in the art. It is intended that the specification and examples be considered as exemplary only, with the true scope and spirit of the disclosure being indicated by the following claims. All references, patents, and patent applications referred to in this application are herein incorporated by reference in their entirety.

What is claimed is:

1. A next generation sequencing-based method for detecting a gene copy number variant (CNV) in a biological sample having one or more genes, the method comprising:
a) obtaining a set of probes containing a number of probes for next generation sequencing (NGS) wherein each probe in the set hybridizes a different segment of the one or more genes;
b) performing next generation sequencing with the set of probes on the biological sample comprising the one or more genes to obtain a sequence read for each probe;
c) creating a normalization baseline for a probe;
d) generating a coverage index for a probe in the set of probes; and
e) determining a difference between the coverage index of the probe and a set confidence interval established from a normal range of coverage index, wherein a CNV is detected where a p-value for the difference between the coverage index of the probe and the set confidence interval is equal to or less than a set threshold.

2. The method of claim 1, wherein the normalization baseline is determined by adding the sequence read of each probe to obtain a total number of sequence reads for the set of probes and dividing the total number of sequence reads for the set of probes by the number of probes in the set of probes.

3. The method of claim 1, wherein the coverage index of the probe is determined by dividing the number of sequence reads obtained for the probe by the normalization baseline.

4. The method of claim 1, wherein the normal range of coverage index is determined by:
a) obtaining one or more biological samples having a normal copy number for each of the one or more genes;
b) performing NGS on the one or more biological samples with the set of probes;
c) adding the sequence reads from each probe in the set to obtain a total number of sequence reads for the set of probes for each of the biological samples;
d) dividing the total number of sequence reads for each of the biological samples by the number of probes in the set of probes to generate a normalization baseline for a probe;
e) calculating a coverage index for each probe in the set of probes for the biological samples by dividing the number of sequence reads obtained for the probe from each biological sample by the normalization baseline to determine a normal range of coverage index.

5. The method of claim 1, wherein the CNV is in an exon.

6. The method of claim 1, wherein the CNV is in an intron.

7. The method of claim 1, wherein the CNV is a duplication.

8. The method of claim 1, wherein the CNV is a deletion.

9. The method of claim 8, wherein the deletion is heterozygous or homozygous.

10. The method of claim 1 further comprising obtaining the biological sample from a patient.

11. The method of claim 1, wherein the biological sample is blood, saliva, other liquid biopsies, or solid tumors.

12. The method of claim 1, wherein the set of probes comprises more than 550 probes.

13. The method of claim 1, wherein the one or more genes comprise genes associated with cancer.

14. The method of claim 1, wherein the one or more genes comprise genes associated with diseases that are linked to germline or somatic genetic CNV.

15. The method of claim 1, wherein the one or more genes comprise BRCA1, BRCA2, APC, MLH1, MSH₂, MSH6, PMS2, EPCAM, TP53, CDH1, STK11, SMAD4, VHL, NF2, PTEN, ATM, AXIN2, BARD1, BLM, BMPR1A, BRIP1, BUB1B, CDK4, CDKN2A, CHEK2, EXO1, FLCN, GREM1, MLH3, MRE11A, MUTYH, NBN, NF1, PALB2, PMS1, POLD1, POLE, RAD50, RAD51C, RAD51D, or TGFBR2.

16. The method of claim 1, wherein the set confidence interval is based on a 99% confidence level.

17. The method of claim 1, wherein the set threshold is 10⁴.

18. A method for detecting a gene copy number variant (CNV) in a biological sample having one or more genes, the method comprising:
a) obtaining a set of probes for next generation sequencing wherein each probe in the set hybridizes a different segment of the one or more genes in the biological sample;
b) performing next generation sequencing with the set of probes on the biological sample to obtain a sequence read for each probe;
c) adding the sequence reads from each probe to obtain a total number of sequence reads for the set of probes;
d) dividing the total number of sequence reads by the number of probes in the set of probes to generate a normalization baseline for a probe;
e) determining a coverage index for the probe in the set of probes by dividing a number of sequence reads obtained for the probe by the normalization baseline; and
f) generating a p-value for a difference between the coverage index of the probe and a set confidence interval established from a normal range of coverage index, wherein a CNV is detected where the p-value is equal to or less than 10⁻⁴.

19. The method of claim 18, wherein the normal range of coverage index is determined by:
a) obtaining one or more biological samples having a normal copy number for each of the one or more genes;
b) performing NGS on the one or more biological samples with the set of probes;
c) adding the sequence reads from each probe to obtain a total number of sequence reads for the set of probes for each of the biological samples;
d) dividing the total number of sequence reads for each of the biological samples by the number of probes in the set of probes to generate a normalization baseline for a probe;
e) calculating a coverage index for each probe in the set of probes for the biological sample by dividing the number of sequence reads obtained for the probe by the normalization baseline to determine a normal range of coverage index.

20. The method of claim 18, wherein the CNV is in an exon.

21. The method of claim 18, wherein the CNV is in an intron.

22. The method of claim 18, wherein the CNV is a duplication.

23. The method of claim 18, wherein the CNV is a deletion.

24. The method of claim 23, wherein the deletion is heterozygous or homozygous.

25. The method of claim 18 further comprising obtaining the biological sample from a patient.

26. The method of claim 18, wherein the biological sample is blood, saliva, other liquid biopsies, or solid tumors.

27. The method of claim 18, wherein the set of probes comprises more than 550 probes.

28. The method of claim 18, wherein the one or more genes comprise genes associated with cancer.

29. The method of claim 18, wherein the one or more genes comprise genes associated with diseases that are linked to germline or somatic genetic CNV.

30. The method of claim 18, wherein the one or more genes comprise BRCA1, BRCA2, APC, MLH1, MSH₂, MSH6, PMS2, EPCAM, TP53, CDH1, STK11, SMAD4, VHL, NF2, PTEN, ATM, AXIN2, BARD1, BLM, BMPR1A, BRIP1, BUB1B, CDK4, CDKN2A, CHEK2, EXO1, FLCN, GREM1, MLH3, MRE11A, MUTYH, NBN, NF1, PALB2, PMS1, POLD1, POLE, RAD50, RAD51C, RAD51D, or TGFBR2.

31. The method of claim 18, wherein the set confidence interval is based on a 99% confidence level.

32. The method of claim 18, wherein the set threshold is 10⁻⁴.

33. An electronic computer system, comprising:
a) one or more processors; and
b) a memory storing one or more programs for execution by the one or more processors, the one or more programs comprising instructions for:
i) analyzing data obtained from next generation sequencing of a biological sample having one or more genes using a set of probes, wherein the data comprises sequence reads for each probe;
ii) creating a normalization baseline for a probe;
iii) generating a coverage index for a probe in the set of probes;
iv) determining a difference between the coverage index of the probe and a set confidence interval established from a normal range of coverage index, wherein a copy number variant is detected where a p-value for the difference is equal to or less than a set threshold.

34. The electronic computer system of claim 33, wherein the coverage index of the probe is determined by dividing the number of sequence reads obtained for the probe by the normalization baseline.

35. The electronic computer system of claim 33, wherein the normal range of coverage index is determined by:
a) obtaining one or more biological samples having a normal copy number for each of the one or more genes;
b) performing NGS on the one or more biological samples with the set of probes;
c) adding the sequence reads from each probe to obtain a total number of sequence reads for the set of probes for each of the biological samples;
d) dividing the total number of sequence reads for each of the biological samples by the number of probes in the set of probes to generate a normalization baseline for a probe;
e) calculating a coverage index for each probe in the set of probes for the biological sample by dividing the number of sequence reads obtained for the probe by the normalization baseline to determine a normal range of coverage index.

36. The electronic computer system of claim 33, wherein the set confidence interval is based on a 99% confidence level.

37. The electronic computer system of claim 33, wherein the set threshold is 10⁻⁴.

38. A method of treating a patient with a targeted therapy, the method comprising:
a) determining if a gene copy number variant (CNV) is present in a biological sample obtained from the patient, comprising the steps of:
i) obtaining a set of probes for next generation sequencing (NGS) wherein each probe in the set hybridizes a different segment of the one or more genes;
ii) performing next generation sequencing with the set of probes on the biological sample comprising the one or more genes to obtain a sequence read for each probe;
iii) creating a normalization baseline for a probe;
iv) generating a coverage index for a probe in the set of probes; and
v) determining a difference between the coverage index of the probe and a set confidence interval established from a normal range of coverage index, wherein a CNV is detected where a p-value for the difference between the coverage index of the probe and the set confidence interval is equal to or less than a set threshold; and
b) administering the targeted therapy to the patient where a CNV is detected in the biological sample.
